(19) United States
(12) Patent Application Publication
(10) Pub. No.: US 2025/0120760 A1
(43) Pub. Date: Apr. 17, 2025

(54) COAXIAL DUAL COMPONENT LOCKING SCREW

(71) Applicant: WHEELHOUSE MEDICAL DEVICE & DESIGN CONSULTING INC., Monroe, NC (US)

(72) Inventor: John SOUZA, Monroe, NC (US)

(21) Appl. No.: 18/914,778

(22) Filed: Oct. 14, 2024

Related U.S. Application Data
(60) Provisional application No. 63/589,860, filed on Oct. 12, 2023.

(51) Int. Cl. A61B 17/86; A61B 17/56

(52) U.S. Cl. CPC A61B 17/8685; A61B 17/863; A61B 2017/564; A61B 2017/8655

(57) ABSTRACT

A coaxial dual component locking screw includes a screw component and a housing component. The screw component includes a thread extending at least a portion of the length from the tip to the head and the housing component includes each having a plurality of wherein the slits of the housing component are adapted to correspond to a pitch of the thread of the screw component. When the screw component and the housing component are engaged, the thread of the screw component protrudes through the slits and provides resistance to migration and pull out of the coaxial dual component locking screw when implemented in a bone.

### REFERENCE TO RELATED APPLICATIONS

This application claims priority to and the benefit of U.S. Provisional Application No. 63/589,860, filed Oct. 12, 2023, which is hereby incorporated by reference in its entirety.

### FIELD OF THE INVENTION

This disclosure is directed to a coaxial dual component locking screw and methods of its use. More specifically, this disclosure is directed to a coaxial dual component locking screw for implantation as a bone screw.

### BACKGROUND OF THE INVENTION

Bone screws are designed to be implanted in bone and have a number of applications. Bone screws may be used to hold two bones together. They may also be used to hold an orthopedic plate, rod or other implant in place to provide stability while a fractured bone heals.

The sacroiliac joint is a joint that is held together by strong ligaments between the sacrum and ilium bones of the pelvis. When the joint is no longer working properly, it may cause pain in the hip and lower back, and surgery to fuse or join the sacrum to the ilium by a bone screw may be advantageous to reduce the pain. An injury or degeneration of the bones of the spinal column may also require surgery to fuse or join by a bone screw two or more vertebrae.

### BRIEF DESCRIPTION OF THE INVENTION

A coaxial dual component locking screw includes a screw component and a housing component. The screw component has a tip at a screw distal end, a head at a screw proximal end, and a thread extending at least a portion from the tip to the head. The housing component includes sidewalls extending from a housing proximal end to a housing distal end with a plurality of slits disposed along the sidewalls. The slits of the housing component are adapted to correspond to a pitch of the thread of the screw component. The housing proximal end is adapted to receive the screw distal end, and when the screw component and the housing component are fully engaged with the screw component extending into the housing component, the thread of the screw component protrudes through the slits and provides resistance to migration and pull out the coaxial dual component locking screw when implemented in a bone.

An implantation method includes inserting a housing component of a coaxial dual component locking screw into a void space adjacent to bone. The housing component includes sidewalls extending from a housing proximal end to a housing distal end with a plurality of slits disposed along the sidewalls. The implantation method also includes screwing a screw component of the coaxial dual component locking screw into the housing proximal end of the housing component. The screw component has a tip at a screw distal end, a head at a screw proximal end, and a thread extending at least a portion from the tip to the head. The slits of the housing component are adapted to correspond to a pitch of the thread of the screw component and receive the thread during the screwing. The thread of the screw component protrudes through the slits after the screwing and provides resistance to removal of the coaxial dual component locking screw from the void space.

### BRIEF DESCRIPTION OF THE DRAWINGS

FIG. 1 shows a perspective view of a screw component disengaged from a housing component of a coaxial dual component locking screw in an embodiment of the present disclosure.

FIG. 2 shows a side view of the screw component of the coaxial dual component locking screw of FIG. 1.

FIG. 3 shows a perspective view of the screw component of FIG. 2.

FIG. 4 shows a top view of the screw component of FIG. 2.

FIG. 5 shows a bottom view of the screw component of FIG. 2.

FIG. 6 shows a perspective view of the housing component of the coaxial dual component locking screw of FIG. 1.

FIG. 7 shows a side view of the housing component of FIG. 6.

FIG. 8 shows a top view of the housing component of FIG. 6.

FIG. 9 shows a bottom view of the housing component of FIG. 6.

FIG. 10 shows a perspective view of the screw component fully engaged with the housing component of the coaxial dual component locking screw of FIG. 1.

FIG. 11 shows a top view of the coaxial dual component locking screw of FIG. 10.

FIG. 12 shows a bottom view of the coaxial dual component locking screw of FIG. 10.

FIG. 13 shows a perspective view of a screw component disengaged from a housing component of a coaxial dual component locking screw in another embodiment of the present disclosure.

FIG. 14 shows a side view of the screw component of the coaxial dual component locking screw of FIG. 13.

FIG. 15 shows a perspective view of the screw component fully engaged with the housing component of the coaxial dual component locking screw of FIG. 13.

FIG. 16 shows a perspective view of a screw component disengaged from a housing component of a coaxial dual component locking screw in another embodiment of the present disclosure.

FIG. 17 shows a side view of the screw component of the coaxial dual component locking screw of FIG. 16.

FIG. 18 shows a perspective view of the screw component of the coaxial dual component locking screw of FIG. 16.

FIG. 19 shows a side view of the screw component partially engaged with the housing component of the coaxial dual component locking screw of FIG. 16.

FIG. 20 shows a perspective view of the screw component fully engaged with the housing component of the coaxial dual component locking screw of FIG. 16.

### DETAILED DESCRIPTION OF THE INVENTION

The general inventive concepts will now be described with occasional reference to the exemplary embodiments of the invention. The general inventive concepts may be embodied in different forms and should not be construed as limited to the embodiments set forth herein. Rather, these embodiments are provided so that this disclosure will be thorough and complete, and will fully convey the scope of the general inventive concepts to those skilled in the art.

As used herein, the singular forms “a,” “an,” and “the” are intended to include the plural forms as well, unless the context clearly indicates otherwise. The term “proximal” as used in connection with any object refers to the portion of the object that is closest to the operator of the object (or some other stated reference point), and the term “distal” refers to the portion of the object that is farthest from the operator of the object (or some other stated reference point).

One of ordinary skill will appreciate that references to positions in the body are merely representative for a particular surgical approach. Further, some references herein are made in the context of the representative images shown in the drawings. Fewer or additional instruments, including generic instruments, may be used according to the preference of the operator. Moreover, references herein to specific instruments are not intended to be limiting in terms of the options for use of other instruments where generic options are available, or according to the preference of the operator.

The present application describes various embodiments of devices for joint and other bone fixation. In some particular embodiments, devices for fixation of the sacroiliac joint are provided. It will be appreciated that the examples and drawings, as shown herein, may be described in reference to use in applications for sacroiliac joint fusion, though the devices as disclosed herein may be used in any of a variety of other orthopedic applications, and may be used alone, or as an adjunct to devices used for other fixation or correction, such as, for example, in spine fusion surgery, to help hold other implants in place.

Provided is a coaxial dual component locking screw that locks in place in contact with bone by two mechanisms to resist rotation and pull-out after implantation.

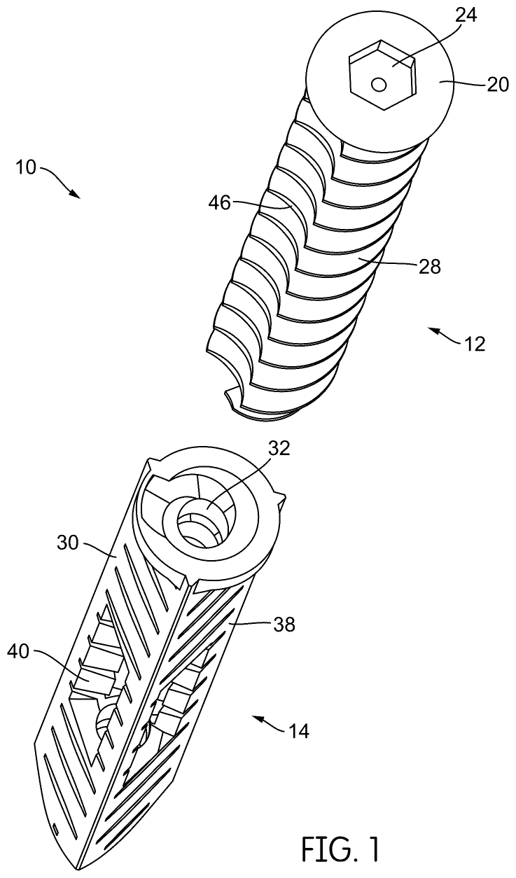

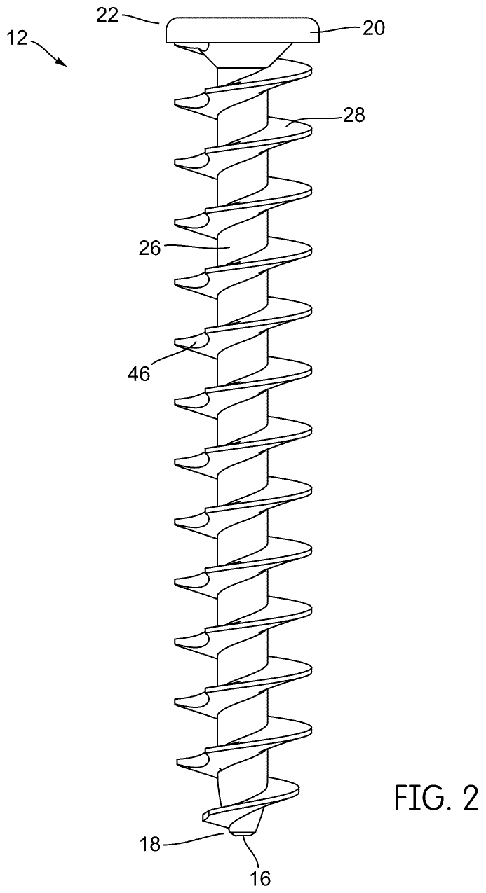

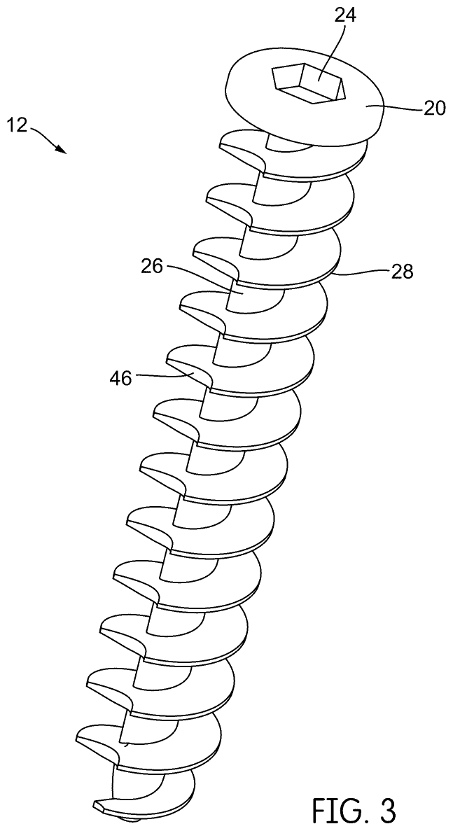

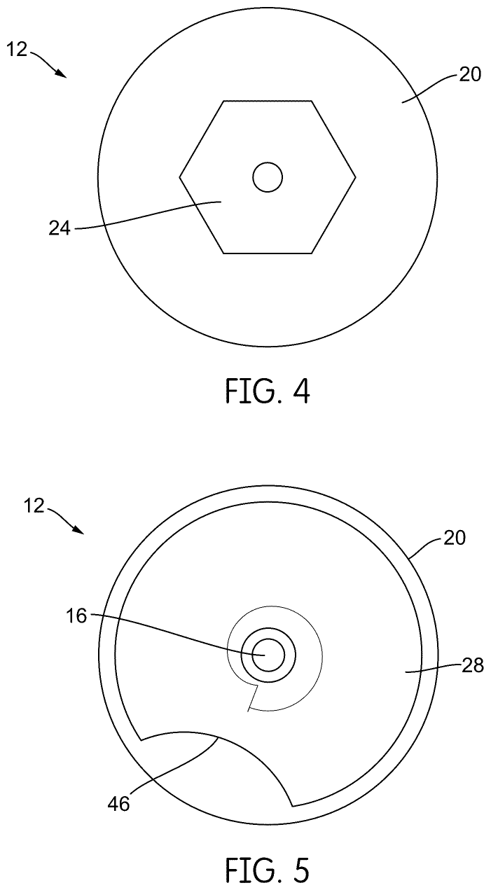

Referring to FIG. 1, a coaxial dual component locking screw 10 includes a screw component 12 and a housing component 14. Referring to FIG. 2, the screw component 12 has a tip 16 at its distal end 18 and a head 20 at its proximal end 22. Although shown with substantially flat upper and lower surfaces and a slight taper toward the upper surface in FIG. 2, the head 20 may alternatively have a cylindrical, hemispherical, spherical, or conical shape. As shown in FIGS. 3-4, the head 20 includes a recess 24 for actuating the screw component 12 with a tool. Although a hexagonal recess for an Allen wrench is shown, the head 20 may alternatively include any other appropriate recess 24, such as, for example, a slot for a Phillips head or a flat head screwdriver. A central shaft 26 of the screw component 12 extends from the head 20 toward the tip 16 with a thread 28 extending around the central shaft 26. The thread 28 is disposed with a pitch. As best shown in FIG. 5, the head 20 has a slightly larger diameter than the thread 28.

In various embodiments, the head 20 has a shape that is one of generally disc shaped with curvate edges, semi-spherical, spherical, hemispherical, cylindrical, conical, and frustoconical. In some specific embodiment as depicted herein, the head 20 has a generally disc shape that slightly tapers from a distal portion of the head and the thread 28 extends from the distal portion of the head 20 towards the tip 16. As depicted, the head has a diameter that is greater than the diameter of the portion of the screw extending from the head 20 to the tip 16. In other embodiments, the head 20 may have a diameter that is the same as the screw excluding the thread 28

In some embodiments, the thread 28 spans the entire length of the screw between the head and the tip. In other embodiments, the thread 28 is spans only a portion of the length of the screw between the head and the tip. In other embodiments, the thread 28 is spans only a portion of the length of the screw between the head and the tip, and the screw portion between the head and the tip includes one or more additional threads each having a thread length, pitch, or both that is different from the thread 28. In some embodiments, the tip of the screw is threaded.

In some embodiments, the thread 28 is continuous along at least a portion of the length of the screw between the head 20 and the tip 16. In some embodiments, the thread 28 is dis-continuous (i.e., interrupted, includes a gap) along the length of the screw between the head 20 and the tip 16. In some embodiments, the pitch of the thread 28 can be constant along at least a portion of the length of the screw between the head 20 and the tip 16. In other embodiments, the pitch of the thread 28 can vary along at least a portion of the length of the screw between the head 20 and the tip 16.

In various embodiments, the thread 28 may be shaped as known in the art, including shapes selected from V-, square-, buttress-, reverse buttress, and combinations of these, and the thread features may be left or right hand oriented.

In various embodiments, the tip 16 may be a rounded tip as illustrated for example in FIG. 1. In other embodiments, the tip 16 may be self-tapping tip or any other suitable tip for an orthopedic bone screw known in the art.

In various embodiments, the screw component 12 may be cannulated and is thus adapted to receive a guidewire for directing placement in bone. In other embodiments, the screw component 12 is not cannulated.

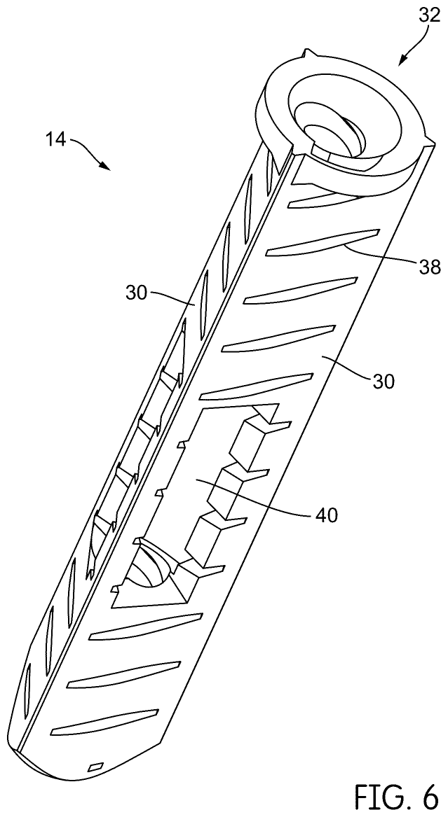

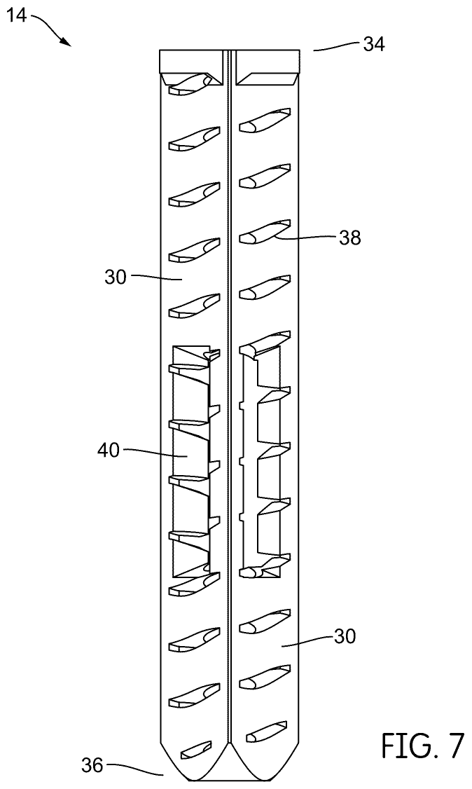

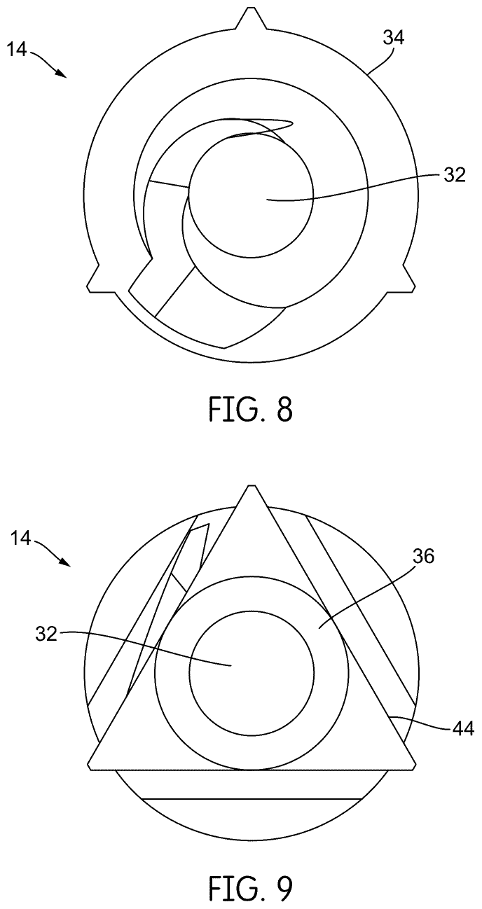

Referring to FIGS. 6-9, the housing component 14 includes sidewalls 30 and a generally cylindrical central opening 32 extending axially through the housing component 14 sized and shaped to receive the central shaft 26 of the screw component 12. As best seen in FIGS. 6-7, the sidewalls 30 have a flat outer surface. Referring to FIG. 7, the housing component 14 extends from a proximal end 34 to a distal end 36. The housing component 14 includes a plurality of slits 38 disposed along the sidewalls 30. The slits 38 of the housing component 14 are adapted to correspond to the pitch of the thread 28. As best shown in FIG. 7, the distal end 36 of the housing component 14 includes a chamfer that provides a taper to the distal end 36.

Referring to FIGS. 6-7, at least one of the sidewalls 30 includes an aperture 40 that is significantly wider than the slits 38 in the sidewall 30 and provides space for bone ingrowth into the footprint of the housing component 14. The aperture 40 may have any appropriate shape, such as, for example, the shape of a slot.

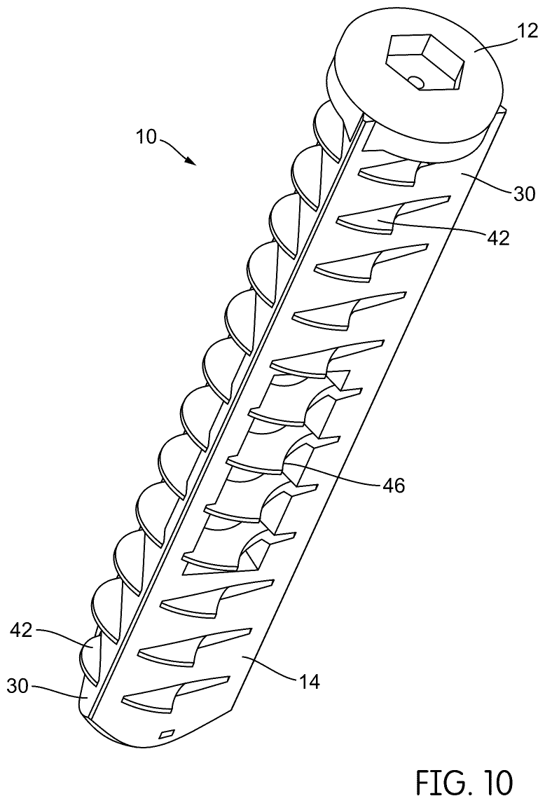

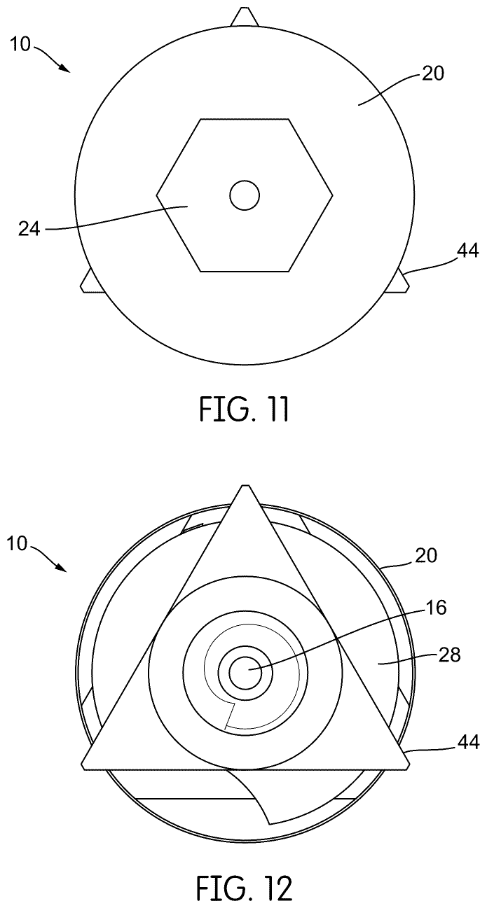

Referring to FIGS. 1 and 10, actuating the screw component 12 drives the central shaft 26 into the central opening 32 of the housing component 14 as the thread 28 engages the slits 38 in the sidewalls 30.

Referring to FIG. 10, when the screw component 12 and housing component 14 are fully engaged, a back surface of the head 20 of the screw component 12 contacts the proximal end 34 of the housing component 14, and the thread 28 of the screw component 12 protrudes through the slits 38 of the housing component 14 to form protrusions 42 that provide resistance to pullout when implemented in a bone. In other embodiments, full engagement may occur without the back surface of the head 20 of the screw component 12 contacting the proximal end 34 of the housing component 14. It will be appreciated that the coaxial dual component locking screw 10 is, or may be adapted to be, suitable for any type of bone grafting.

As best seen in FIG. 11, portions of the cross section 44 of the housing component 14 extend radially farther than the outer diameter of the head 20 of the screw component 12.

As best seen in FIG. 12, the housing component 14 has a substantially triangular cross section 44 that is a generally rectilinear along the central axis.

A coaxial dual component locking screw 10 of the present disclosure may have wide applications, including but not limited to, surgical applications, such as, for example, joining bones, such as at sacroiliac joints or vertebral pedicles or other spinal anatomies, securing implants into bones, or dental applications.

In some embodiments, the coaxial dual component locking screw 10 is implemented during a surgery. The housing component 14 is inserted into the void space at the surgical site, where at least a portion of the edge of the void space is defined by bone. In some embodiments, the void space is sized and shaped to receive the housing component 14. In some embodiments, the void space is formed prior to inserting the housing component 14. In some embodiments, the void space is formed by removal of bone material. The screw component 12 is then screwed into the housing component 14 to provide the coaxial dual component locking screw 10 at the implantation site whereupon the protrusions 42 of the screw component 12 are exposed to engage the bone.

In some embodiments, the void space is oriented along the joint and parallel to the surfaces of two bones to be stabilized, joined, or fused. In some embodiments, the bones include two adjacent vertebrae.

In some embodiments, the void space is oriented across the joint and perpendicular to the surfaces of two bones to be stabilized, joined, or fused. In some embodiments, the bones include an ilium and a sacrum adjacent the ilium.

In some embodiments, one or more features of the coaxial dual component locking screw 10 are customized for a specific application or surgical location.

In some embodiments, the customization includes a customized material of the coaxial dual component locking screw 10. The screw component 12 and the housing component 14 of a coaxial dual component locking screw 10 may be formed of any appropriate material. The screw component 12 and the housing component 14 of a coaxial dual component locking screw 10 may be formed of the same material or different materials. Appropriate implantable materials for the components may include, but are not limited to, titanium, titanium alloy, polyether ether ketone (PEEK), cobalt, stainless steel, or combinations thereof.

The material of the screw component 12 and the housing component 14 may be non-porous or have any porosity suitable to promote bone ingrowth into the component while maintaining the structural integrity of the coaxial dual component locking screw 10.

In some embodiments, the customization includes a surface coating and/or a surface texturing on all or a portion of the surface of the screw component 12 and/or the housing component 14. For example, all or a portion of the outer and inner surfaces of the housing component 14 may be adapted with surface shaping and/or texturing to create variations in material composition and surface roughness to thereby encourage bone ingrowth or on-growth, which may include one or more surface features including ribbing, grooves, knurling, or surface texturing, or coatings of hydroxyapatite (HA) to PEEK or titanium surfaces, or titanium to PEEK implant surfaces, or texturing by media blasting to prepare a roughened micro- or nano-topography on titanium to promote bioactivity and bone on-growth, or plasma treatment, to alter surface chemistry and nano-topography of PEEK for improved wettability.

In some embodiments, the customization includes a customized thread pitch. The thread 28 of the screw component 12 may have a single pitch or multiple different pitches. In some embodiments, the screw component 12 includes two or more partial threads 28, which may have the same or different pitches. In some embodiments, different pitches permit the coaxial dual component locking screw 10 to apply a compacting or a stretching force to the bone.

In some embodiments, the customization includes a taper to the screw component 12 or the housing component 14. In some embodiments, the central shaft 26 and thread 28 of the screw component 12 have a uniform diameter along its length. In other embodiments, the screw component 12 has a tapered proximal end 22 and/or distal end 18. In some embodiments, the housing component 14 has a generally rectilinear cross section 44 along the central axis. In other embodiments, the housing component 14 has a tapered proximal end 34 and/or a tapered distal end 36.

In some embodiments, the customization includes a customized housing cross section 44. In some embodiments, the housing component 14 has a generally triangular cross section 44 transverse to the central axis. The housing component 14 may alternatively have other cross sections 44, such as, for example, rectangular, circular, or combinations thereof.

In some embodiments, the customization includes customized relative lengths of the screw component 12 and the housing component 14. In some embodiments, the tip 16 of the screw component 12 aligns or substantially aligns with the distal end 36 of the housing component 14, when the screw component 12 and the housing component 14 of the coaxial dual component locking screw 10 are fully engaged with each other. In other embodiments, the tip 16 of the screw component 12 extends past the distal end 36 of the housing component 14, when the screw component 12 and the housing component 14 are fully engaged with each other. In other embodiments, the distal end 36 of the housing component 14 extends past the tip 16 of the screw component 12, when the screw component 12 and the housing component 14 are fully engaged with each other.

In some embodiments, the customization includes a customized thread width that provides a varying contour to the outer edge of the thread. In some embodiments, the width of the thread 28 as measured from the central axis of the screw component 12 is constant along the entire length of the thread 28. In other embodiments, the width of the thread 28 tapers over at least a portion from the distal end 18 to the proximal end 22 or from the proximal end 22 to the distal end 18. In other embodiments, the width of the thread 28 is irregular along at least a portion of the thread 28. Referring to FIGS. 1-3 and 10, the irregularity 46 includes a concavely curved section along one radial location down the length of the thread 28. Referring to FIG. 10, the irregularity 46 affects the shape of some of the protrusions 42 in the assembled coaxial dual component locking screw 10. Other irregularities may include, but are not limited to, convexly curved sections, saw-toothed sections, or randomized sections. Thus, in some embodiments as depicted in the referenced drawing FIG. 10, the concavity results in limited or the absence of protruding threads from the slits in the housing.

In some embodiments, the customization includes a customized thread length. In some embodiments, the thread 28 extends substantially from the head 20 to the tip 16 of the screw component 12. In other embodiments, a portion of the length of the screw component 12 does not include a thread 28.

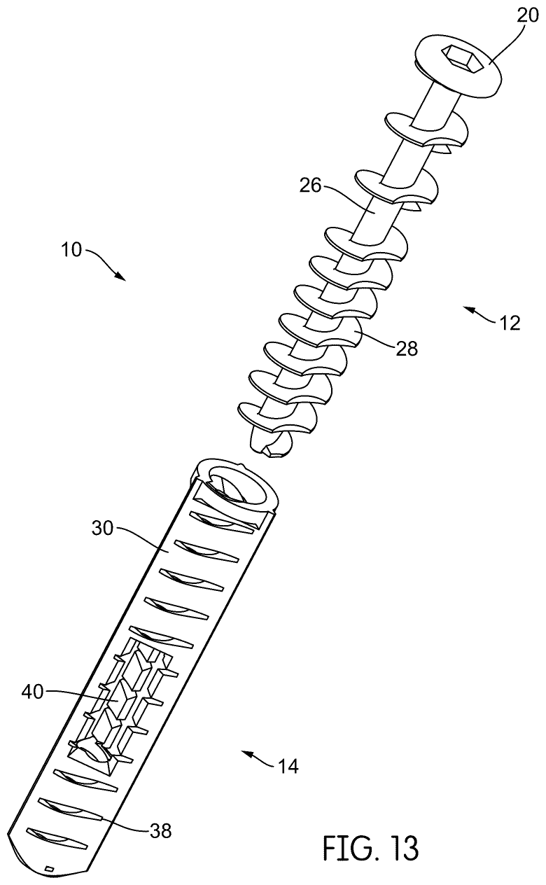

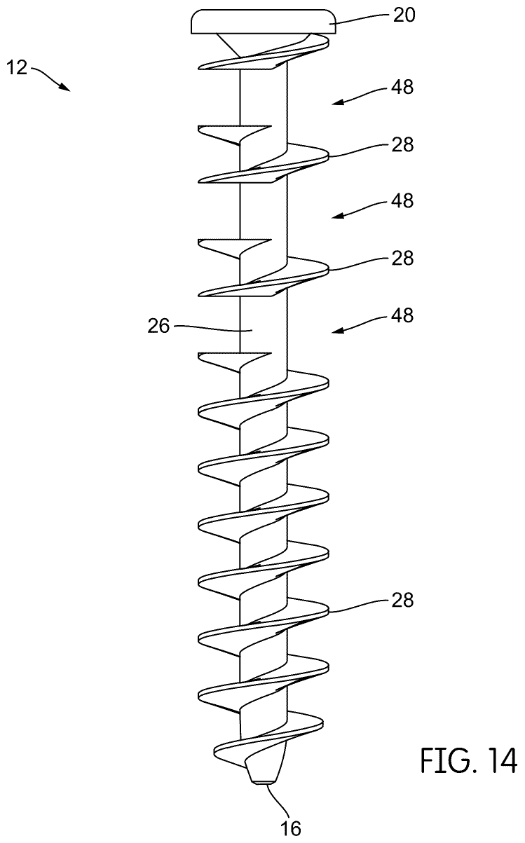

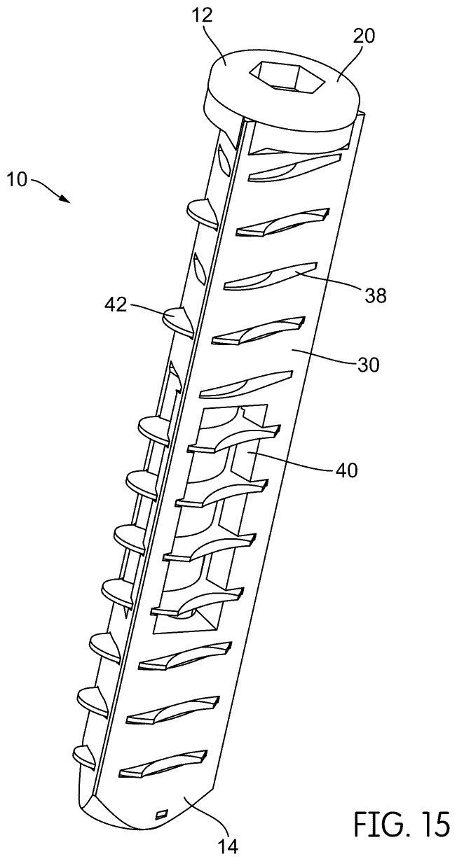

Referring to FIGS. 13-15, the screw component 12 includes two discontinuities 48 in the thread 28 along the central shaft 26 of the screw component 12. These discontinuities 48 provide variation to the pattern of protrusions 42 in the assembled coaxial dual component locking screw 10 and may also permit the screw component 12 to apply different axial forces on the housing component 14 in the assembled coaxial dual component locking screw 10.

In some embodiments, the customization includes one or more apertures 40 in the housing component 14. In some embodiments, the housing component 14 includes no apertures 40. In other embodiments, the housing component 14 includes at least one aperture 40 in at least one sidewall 30. An aperture 40 may have any length along the axis of the housing component 14. In some embodiments, a sidewall 30 may have more than one aperture 40. In some embodiments, an aperture 30 extends the entire length of one sidewall 40 of the housing component 14 from the distal end 36 to the proximal end 34. In some embodiments, each sidewall 30 includes an aperture 40 and the apertures 40 are axially aligned with each other.

In some embodiments, the coaxial dual component locking screw 10 is customized to provide a compressing force to the bone. In some such embodiments, the housing component 14 includes a central section where all of the sidewalls 30 include an aperture 40 and the screw component 12 includes a discontinuity 48 between the head 20 and where the thread 28 starts that is free of thread 28. The screw component 12 is screwed into the housing component 14 until the back of the head 20 contacts the proximal end 34 of the housing component 14. At this point, the start of the thread 28 closest to the head 20 is in the central section of the housing component 14, and further screwing into the housing component 14 causes the central section to buckle as the distal end 36 of the housing component 14 is pulled toward the proximal end 34.

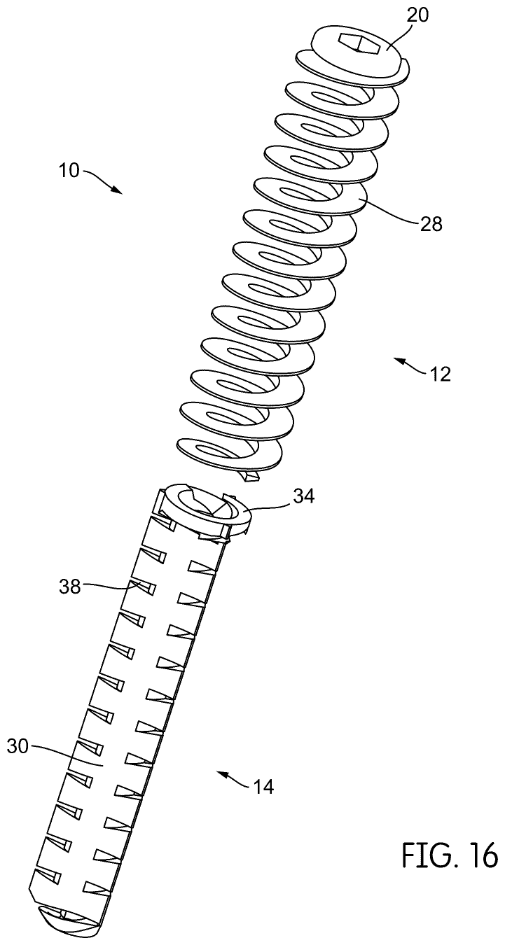

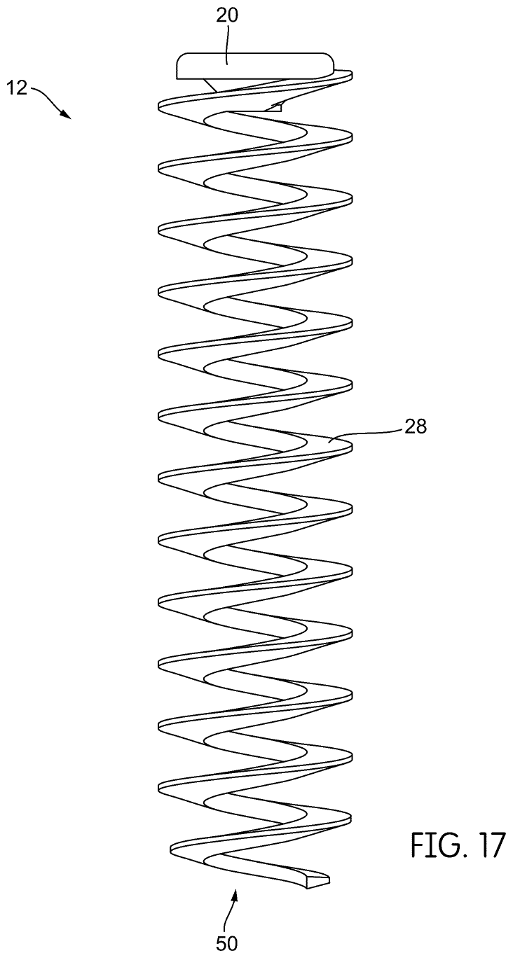

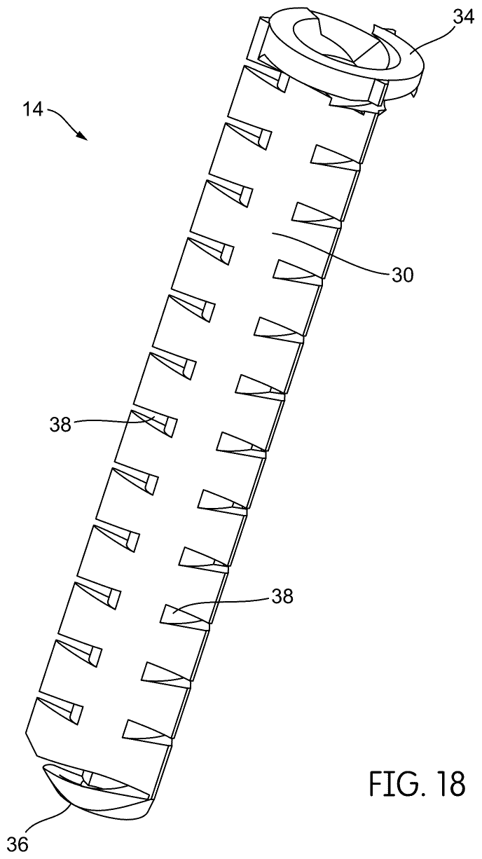

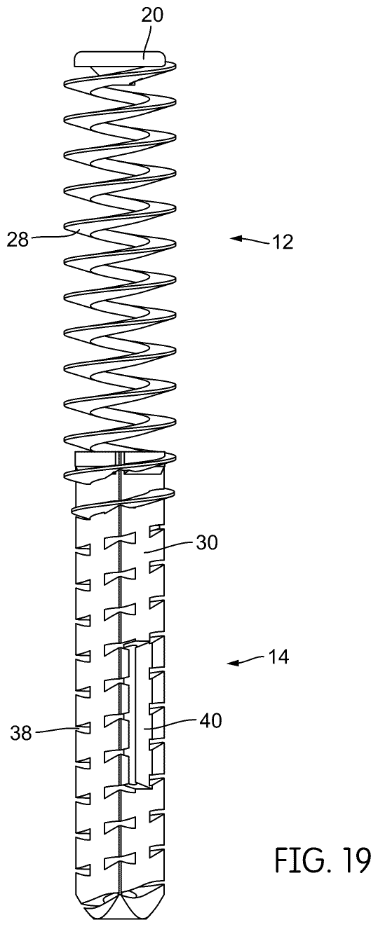

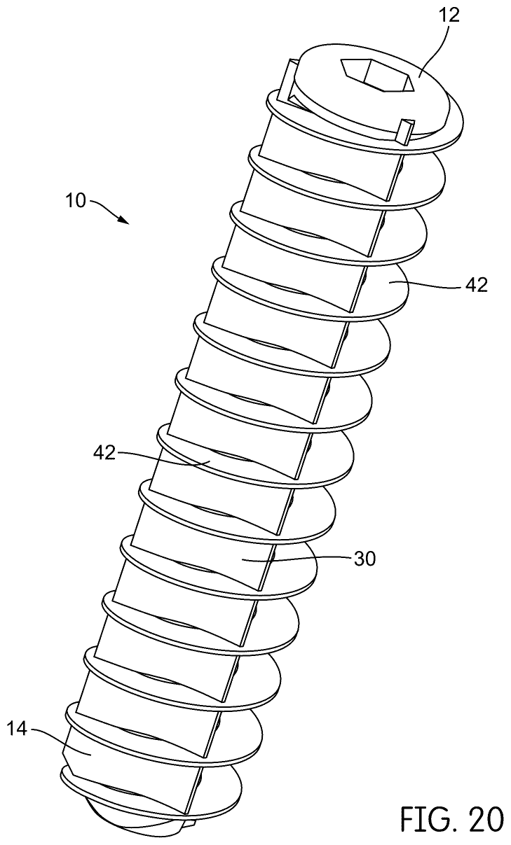

In some embodiments, the coaxial dual component locking screw 10 is customized to include a screw component 12 without a central shaft 26 and a housing component without a central opening 32. Referring to FIGS. 16-17, the screw component 12 includes a head 20 and a thread 28, but the thread 28 is formed around a central opening 50 of the screw component. Referring to FIG. 18, the proximal end 34 of the housing component 14 is adapted to receive the distal end of the screw component 12 and the sidewalls 30 of the housing component 14 includes slits 38 to receive the thread 28, though in contrast to other embodiments, the housing component 14 does not include a central opening 32. Referring to FIGS. 19 and 20, the thread 28 engages the slits 38 as the screw component 12 is screwed into the housing component 14. Referring to FIG. 19, the housing component 14 may include an aperture 40 in at least one sidewall 30 of the housing component 14.

While the invention has been described with reference to one or more exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention but that the invention will include all embodiments falling within the scope of the appended claims. In addition, all numerical values identified in the detailed description shall be interpreted as though the precise and approximate values are both expressly identified.

Table of Reference Numerals

| | |
|---|---|
| coaxial dual component locking screw | 10 |
| screw component | 12 |
| housing component | 14 |
| screw component tip | 16 |
| screw component distal end | 18 |
| screw component head | 20 |
| screw component proximal end | 22 |
| recess | 24 |
| central shaft | 26 |
| thread | 28 |
| sidewall | 30 |
| housing central opening | 32 |
| housing component proximal end | 34 |
| housing component distal end | 36 |
| slit | 38 |
| aperture | 40 |
| protrusion | 42 |
| cross section | 44 |
| irregularity | 46 |
| discontinuity | 48 |
| screw central opening | 50 |

What is claimed is:

1. A coaxial dual component locking screw comprising:
a screw component having a length, a tip at a screw distal end, a head at a screw proximal end, and a thread extending along at least a portion of the length of the screw component from the tip to the head; and
a housing component having a length and comprising a central opening and sidewalls extending from a housing proximal end to a housing distal end with a plurality of slits disposed along the sidewalls;
wherein the slits of the housing component are adapted to correspond to a pitch of the thread of the screw component; and
wherein the housing proximal end is adapted to receive the screw distal end, and when the screw component and the housing component are fully engaged with the screw component extending into the housing component, the thread of the screw component protrudes through the slits to provide resistance to migration and pull out the coaxial dual component locking screw when implemented in a bone.

2. The coaxial dual component locking screw of claim 1, wherein the housing component has a triangular cross section perpendicular to an axis of the central opening.

3. The coaxial dual component locking screw of claim 1, wherein at least one of the sidewalls includes an aperture extending into the housing component.

4. The coaxial dual component locking screw of claim 1, wherein each of the sidewalls includes an aperture extending into the housing component.

5. The coaxial dual component locking screw of claim 1, wherein one of the sidewalls includes an aperture extending into the housing component, the aperture extending along at least a portion of the length of the housing component from the housing proximal end to the housing distal end.

6. The coaxial dual component locking screw of claim 1, wherein the thread has a varying contour at an outer edge of the thread.

7. The coaxial dual component locking screw of claim 6, wherein the varying contour defines a shape of at least one of the protrusions.

8. The coaxial dual component locking screw of claim 1, wherein the screw component includes a central shaft extending to the screw distal end and the housing component includes a central opening sized and shaped to receive the central shaft.

9. The coaxial dual component locking screw of claim 8, wherein the thread includes at least one discontinuity along the central shaft of the screw component.

10. The coaxial dual component locking screw of claim 1, wherein the screw component includes a central opening defined by the thread.

11. The coaxial dual component locking screw of claim 1, wherein the head includes a recess sized to receive a tip of a tool for rotating the screw component around a central axis of the screw component.

12. An implantation method comprising:
inserting a housing component of a coaxial dual component locking screw into a void space adjacent to bone, the housing component comprising sidewalls extending from a housing proximal end to a housing distal end with a plurality of slits disposed along the sidewalls; and
screwing a screw component of the coaxial dual component locking screw into the housing proximal end of the housing component, the screw component having a tip at a screw distal end, a head at a screw proximal end, and a thread extending at least a portion from the tip to the head;
wherein the slits of the housing component are adapted to correspond to a pitch of the thread of the screw component and receive the thread during the screwing; and
wherein the thread of the screw component protrudes through the slits after the screwing and provides resistance to removal of the coaxial dual component locking screw from the void space.

13. The implantation method of claim 12 further comprising forming the void space prior to inserting the housing component.

14. The implantation method of claim 13, wherein the forming comprises removal of bone material.

15. The implantation method of claim 13, wherein the void space is shaped to receive the housing component.

16. The implantation method of claim 12, wherein the void space is between two bones to be joined.

17. The implantation method of claim 12, wherein the void space extends from a first bone to a second bone to be joined.

18. The implantation method of claim 12, wherein the bone includes two adjacent vertebrae.

19. The implantation method of claim 12, wherein the bone includes an ilium and a sacrum adjacent the ilium.
